(19) United States
(12) Patent Application Publication
(10) Pub. No.: US 2020/0205853 A1
(43) Pub. Date: Jul. 2, 2020

(54) Arthroscopic Cannula with Removable Fluid Seal System

(71) Applicant: Cannuflow, Inc., Campbell, CA (US)

(72) Inventor: Theodore R. Kucklick, Campbell, CA (US)

(73) Assignee: Cannuflow, Inc., Campbell, CA (US)

(21) Appl. No.: 16/729,130

(22) Filed: Dec. 27, 2019

Related U.S. Application Data
(60) Provisional application No. 62/785,411, filed on Dec. 27, 2018.

(51) Int. Cl. A61B 17/34; A61B 17/00

(52) U.S. Cl. CPC A61B 17/3421; A61B 2017/3445; A61B 17/00234

(57) ABSTRACT

A cannula with a removable and replaceable fluid seal system for use during arthroscopic surgery.

This application claims priority to U.S. Provisional Patent Application 62/785,411 filed Dec. 27, 2018.

### FIELD OF THE INVENTIONS

The inventions described below relate to the field of arthroscopic surgery and, more specifically, to a cannula seal system.

### BACKGROUND OF THE INVENTIONS

Arthroscopy is a minimally invasive procedure for treating joint pathology and is a superior alternative to open joint arthrotomy. Arthroscopy has the advantage of less disruption to the joint tissues and potentially faster healing. The scope of joints and pathologies that can be treated with arthroscopy has grown dramatically, and now includes hip, spine, and small joint procedures in addition to the traditional knee and shoulder procedures. However, arthroscopy remains a technically demanding procedure, and new instrumentation and procedures are constantly being developed.

Instrument cannulas are used to minimize tissue trauma during surgical instrument insertion into a surgical site. Since arthroscopic surgery is performed in a fluid environment, cannulas typically have a fluid seal disposed across the cannula's proximal opening so as to prevent the disruptive backflow of fluid through the cannula. Ideally, the fluid seal should be able to do three things well. First, it should be able to pass an instrument easily therethrough so that the instrument can reach the surgical site from a point outside the body. Second, it should be able to establish an effective fluid seal about an instrument inserted into the cannula, and maintain it while the instrument is in use. Third, it should be able to establish an effective fluid seal across the cannula's proximal opening when no instrument is in the cannula. Effective fluid sealing of the cannula remains a problem. In addition, a fluid sealing system that can be easily removed and replaced as required is needed.

### SUMMARY

The systems and methods described below provide for a removable fluid seal system to hold fluid in the joint and prevent fluid leakage. The fluid seal is effective in preventing backflow and maintaining fluid pressure in the joint during arthroscopic procedures. The seal system may be opened and the seal assembly removed during a procedure to pass items such as a biologic repair construct, stem cells, diced cartilage or tissue adhesives, and then the seal assembly may be replaced onto the cannula proximal section to continue with the arthroscopic procedure and pass instruments through the seal and into the surgical site. A retaining strap ensures the seal assembly is not mislaid during surgery. The system can be used for all types of arthroscopy including knee, shoulder, hip, small joint, and spine procedures.

### BRIEF DESCRIPTION OF THE DRAWINGS

FIG. 1 illustrates the cannula with a removable fluid seal system utilizing a cannula driver.

FIG. 2 illustrates the cannula system of FIG. 1 in the open position.

FIG. 3 illustrates the cannula system of FIG. 1 in the closed position.

FIG. 4 illustrates a top view of a seal system.

FIG. 5 illustrates a cross sectional view of the seal system of FIG. 4.

### DETAILED DESCRIPTION OF THE INVENTIONS

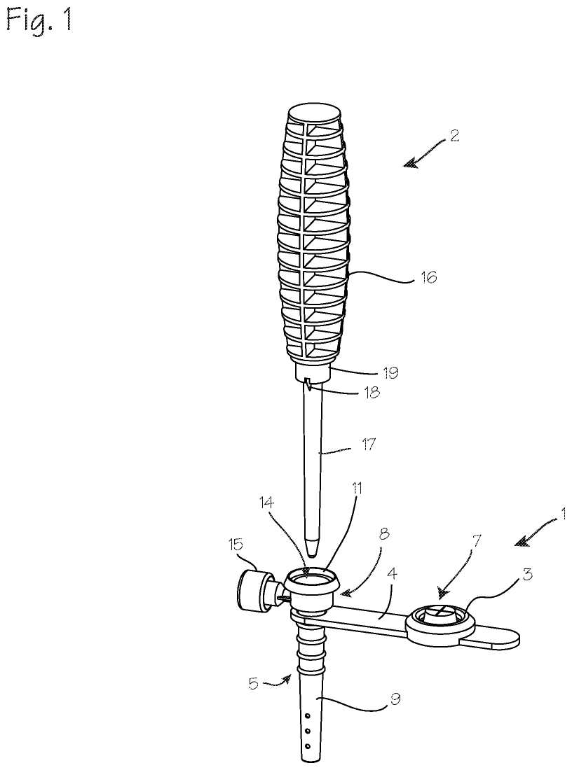

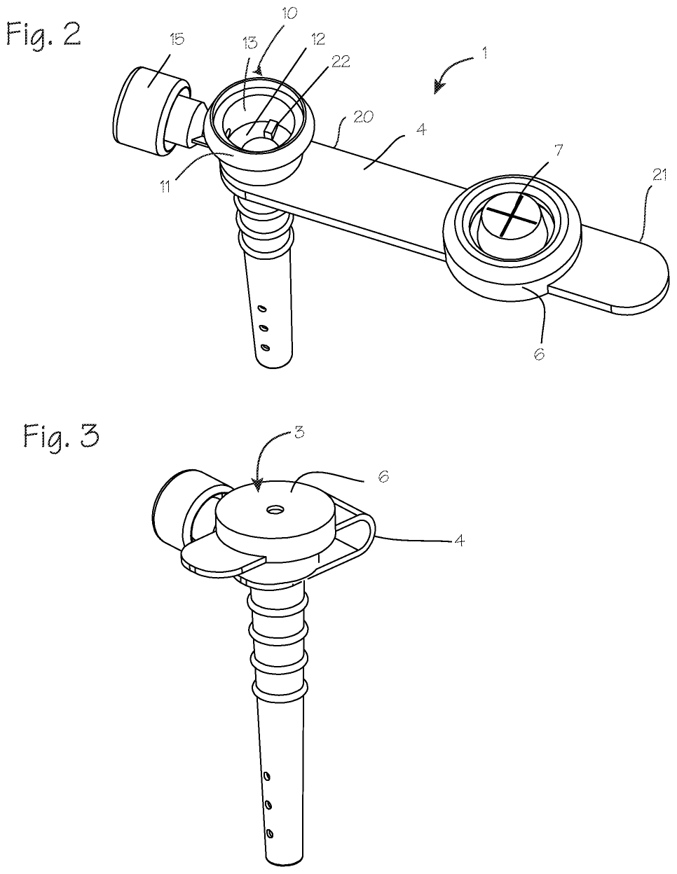

FIG. 1 illustrates the cannula with a removable fluid seal system 1 and a cannula driver 2. The seal system 1 comprises a seal assembly 3 contained on a tether or strap 4 that projects from a cannula 5. The seal assembly 3 combines a zero seal (also known as a wiper seal or backup seal) 6 (shown in FIG. 3) and a slit seal (also known as a septum seal) 7 into a unitary assembly, wherein the slit seal 7 is disposed on top of the zero seal 6 in the open position (FIG. 2) and below the zero seal 6 in the closed position (FIG. 3) when the seal is secured over the proximal opening of the cannula tube. The cannula comprises a proximal section 8 and a distal insertion section 9. The cannula's proximal section 8 includes a top end 10 (shown in FIG. 2) with a flexible flange 11, a bottom end 12 (shown in FIG. 2), and a side wall 13 (shown in FIG. 2) defining a bore 14 extending from the top end to the bottom end, wherein the bore 14 receives the seal assembly 3 when the cannula system is in the closed position (FIG. 3). The proximal section may further include a fluid side port 15 to allow fluid inflow and outflow from the surgical site.

The cannula driver 2 includes a handle 16 connected to a rigid shaft or obturator 17. The driver further includes slots 18 that engage mating tabs (item 22 in FIG. 2) disposed within the bore of the cannula proximal section (further detailed in FIG. 2). The slots 18 are disposed at the point of connection between the handle and the rigid shaft, and may be disposed in the shaft 17 or a collar 19. The collar may be sized to match the bottom 12 of the proximal section, so that the bottom 12 serves as a proximally facing shoulder which limits insertion of the driver into the cannula.

FIG. 2 illustrates the cannula system of FIG. 1 in the open position. The flexible strap 4 has a first end 20 and a second end 21. The strap first end 20 is operably connected to the cannula 5. The strap projects a distance from the cannula and contains the seal assembly 3 disposed on the strap second end 21. The seal assembly 3 comprises two seals, such as a cross slit seal 7 proximate a zero seal 6. The fluid seal system is made of a flexible or resilient material having a high resistance to cutting or tearing as well as good lubricity (polyisoprene or silicone are suitable). The extending lip fits axially into the bore of the cannula in a slight press fit.

The proximal section bottom end 12 further comprises a plurality of tabs 22 sized and dimensioned to engage with the slots 18 of the rigid driver. The driver slots 18 receive and engage with the tabs 22. This engagement feature allows use of the driver to rotate the cannula to facilitate insertion, and these first and second engagement features are configured to rotationally lock the driver to the cannula. The tabs 22 may be rectangular, hexagonal, or other shape so long as the slots 18 are correspondingly shaped and mateable. The opposite configuration is also possible: the driver can have tabs and the cannula can have slots. Also, the driver collar 19 can be hexagonal (rectangular or other) and the cannula proximal section bottom 12 be shaped like a hexagonal socket (rectangular or other).

FIG. 3 illustrates the cannula system of FIG. 1 in the closed position. The strap 4 folds back to allow the seal assembly 3 to snap into the bore 14 of the cannula proximal section to achieve the closed position and inhibit fluid leakage from the inflated joint. As show, the zero seal 6 is now on top with the cross slit seal 7 underneath (not shown).

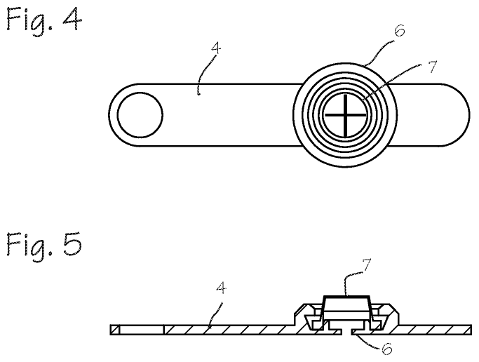

FIG. 4 illustrates a top view of a seal system and FIG. 5 illustrates a cross sectional view of the seal system of FIG. 4. The seal assembly combines the zero seal 6 and the cross slit seal 7 into one unit. The strap 4 of the seal assembly may be molded as a single unit with the seal assembly 3, advantageously ensuring that the seal parts are not mislaid during surgery and are easily replaced. The seal system may be opened and the seal assembly removed from the proximal section 8 of the cannula during a procedure to pass items such as a biologic repair construct, stem cells, diced cartilage or tissue adhesives, and then the seal assembly may be replaced onto the cannula proximal section to continue with the arthroscopic procedure and pass instruments through the seal and into the surgical site.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

I claim:

1. A cannula and driver assembly comprising:
a driver, said driver comprising a handle and an obturator, said driver further comprising a first engagement feature at the proximal end of the obturator; and
a cannula, said cannula comprising a proximal section and a distal section, said proximal section further comprising a second engagement feature configured to engage the first engagement feature when the driver is inserted into the cannula, said first and second engagement features configured to rotationally lock the driver to the cannula.

2. The assembly of claim 1 further comprising:
a seal system, said seal system comprising a seal assembly and a strap, said seal assembly comprising a first seal proximate a second seal, said strap having a first end and a second end, said first end connected to the cannula, said seal assembly disposed on the strap second end, wherein said seal assembly is configured to releasably mate with the cannula proximal section.

3. The assembly of claim 2 wherein the first seal is a cross slit seal and the second seal is a zero seal.

4. A cannula and seal assembly comprising:
a cannula, said cannula comprising a proximal section and a distal section, said proximal section having a top end, a bottom end, and a side wall, said top end, bottom end and side wall defining a bore extending from the top end to the bottom end; and
a seal system, said seal system comprising a seal assembly and a strap, said seal assembly comprising a first seal proximate a second seal, said strap having a first end and a second end, said first end connected to the cannula where the proximal section joins the distal section, said seal assembly disposed on the strap second end, wherein said seal assembly is configured to releasably mate with the bore of the cannula proximal section.

5. The assembly of claim 4 wherein the first seal is a cross slit seal and the second seal is a zero seal.
